(19) United States
(12) Patent Application Publication
(10) Pub. No.: US 2019/0060506 A1
(43) Pub. Date: Feb. 28, 2019

(54) Elafin Incorporated Biomaterials for the Treatment of Chronic Tissue Ulcers

(71) Applicant: The Board of Trustees of the Leland Stanford Junior University, Stanford, CA (US)

(72) Inventors: Mohamed R. Ahmed, San Jose, CA (US); Jayakumar Rajadas, Palo Alto, CA (US); Mohammed Inayathullah Nazir Ahmed, Santa Clara, CA (US); Wenchao Sun, Palo Alto, CA (US); Mark R. Nicolls, Palo Alto, CA (US)

(73) Assignee: The Board of Trustees of the Leland Stanford Junior University, Stanford, CA (US)

(21) Appl. No.: 16/107,941

(22) Filed: Aug. 21, 2018

Related U.S. Application Data
(60) Provisional application No. 62/548,858, filed on Aug. 22, 2017.

(51) Int. Cl. A61L 15/22; A61L 15/44; A61L 15/42; A61L 15/58

(52) U.S. Cl. CPC A61L 15/225; A61L 15/44; A61L 2430/34; A61L 15/58; A61L 15/425

(57) ABSTRACT

The present disclosure provides methods and apparatuses for treating tissue ulcers. The apparatuses include elafin protein incorporated into a biocompatible matrix that allows controlled release of the elafin protein to the wound. The biocompatible matrix may be made of biological polymers such as collagen.

### CROSS REFERENCE TO RELATED APPLICATIONS

This application claims the benefit under 35 U.S.C. § 119(e) of U.S. Provisional Application Ser. No. 62/548,858, filed Aug. 22, 2017, the content of which is incorporated by reference in its entirety into the present disclosure.

### SEQUENCE LISTING

The instant application contains a Sequence Listing which has been submitted in ASCII format via EFS-Web and is hereby incorporated by reference in its entirety. Said ASCII copy, created on Aug. 6, 2018, is named S17-045_ST25.txt and is 928 bytes in size.

### BACKGROUND

Chronic tissue ulcers pose a great challenge to physicians treating the patients. Chronic tissue ulcers caused by diabetes, pressure ulcers, ulcers resulting from arterial and venous insufficiency are a burden for the patients and expensive to treat. Worldwide there are more than 350 million people affected by diabetes alone and about a quarter of the affected population develops foot ulcers in their life time. Foot ulcers are hard to manage once they are formed which results in non-traumatic amputations in their lifetime with an estimated 67% of the patients affected by diabetes undergo this traumatic experience.

Chronic wounds are a major problem to treat and are the result of the failure of the orchestrated events at the cellular level. Wound healing involves activation of many types of cells in the wound area including neutrophils, macrophages, fibroblasts, monocytes, keratinocytes and endothelial cells. The wound healing process initiated by hemostasis progress through a set of other important phenomenon including inflammation, proliferation, and remodeling to regenerate the tissue. Hemostasis at the wound site by the formation of fibrin fibrils generated by thrombin mediated cleavage of fibrin sets stage for the neutrophils to be recruited at the wound site. Neutrophils destroy the pathogenic organisms at the wound site followed by the recruitment of macrophages which engulf the debris and dead cells. Slowly other cell types including fibroblasts and keratinocytes proliferate to dissolve the clot and to form the epidermis respectively. Fibroblasts and myofibroblasts secrete collagen, fibronectin and other extracellular matrix proteins that form granulation tissue resulting in the development of vascularization, re-epithelialization and contraction of the granulation tissue to close the wound.

Chronic wounds do not follow the well-orchestrated phases of healing and often result in defective or delayed regulation of the inflammatory phase and fails to progress through normal wound healing process. The other issues with chronic wounds are the local tissue hypoxia, repetitive trauma, infections combined with impaired cellular responses that perpetuate a deleterious cycle preventing the progression of normal healing process. On the other hand, the high levels of mitogenic activity and cell proliferation is absent in the chronic wounds often resulting in the disruption of the delicate balance between pro-inflammatory cytokines, chemokines, proteases and their inhibitors that exists in normal wounds. As a result, the wound fails to close within a physiologically appropriate time frame. The delayed wound healing also exacerbates scarring due to the prolonged inflammation phase. The excessive infiltration of neutrophils is manifested as the causative agent leading to the overproduction of ROS, causing direct damage to the ECM, cell membrane and premature senescence. The neutrophils also release serine proteases such as elastase and MMPs like collagenase (MMP-8). The secreted elastase degrades important growth factors such as PDGF and TGF while collagenase degrades and inactivates components of the ECM.

### SUMMARY

The experimental examples here demonstrate that elafin, an elastase inhibitor, was effective in promoting wound healing. More interestingly, when the elafin as incorporated into a collagen sponge which was applied to the wound, the wound healing effectiveness was significantly improved.

In accordance with one embodiment of the present disclosure, therefore, provided is a wound dressing, comprising an effective amount of an elafin protein dispersed in a biocompatible matrix. In some embodiments, the elafin protein comprises an amino acid sequence of SEQ ID NO: 1 or an amino acid sequence that has at least 90% sequence identity to SEQ ID NO: 1 and is capable of inhibiting elastase.

In some embodiments, the biocompatible matrix comprises a collagen. In some embodiments, the collagen is type 1 collagen. In some embodiments, the biocompatible matrix comprises from about 5 mg/cm³ to about 100 mg/cm³ collagen. In some embodiments, the biocompatible matrix comprises from about 15 mg/cm³ to about 30 mg/cm³ type 1 collagen.

In some embodiments, the wound dressing comprises from about 20 μg/cm² to about 500 μg/cm² elafin protein. In some embodiments, the wound dressing comprises from about 50 μg/cm² to about 200 μg/cm² elafin protein. In some embodiments, the elafin protein is lyophilized.

Also provided, in one embodiment, is a wound healing apparatus comprising a wound dressing of the present disclosure disposed on a supporting material. In some embodiments, the supporting material is an adhesive bandage.

Yet another embodiment of the disclosure provides a method of preparing a wound dressing of the present disclosure, comprising loading a solution of the elafin protein to the biocompatible matrix and drying biocompatible matrix.

Methods of using the wound dressing and wound healing apparatuses are also provided. In one embodiment, provided is a method of improving the healing of a wound, comprising applying a wound dressing or a wound healing apparatus of the disclosure on the wound. In some embodiments, the wound comprises an ulcer. In some embodiments, the wound comprises chronic ulcer related to a diabetic condition.

### BRIEF DESCRIPTION OF THE DRAWINGS

FIG. 1 presents pictures showing the wound creation in db/db diabetic mice and application of standard gauze and collagen bandage dressings in accordance with various embodiments.

FIG. 2 shows photomicrographs of wound healing studies in the mice at day 7 post wound creation in accordance with various embodiments.

FIG. 3 shows photomicrographs of wound healing studies in the mice at day 14 post wound creation in accordance with various embodiments.

FIG. 4 shows photomicrographs of wound healing studies in the mice at day 21 post wound creation in accordance with various embodiments.

FIG. 5 presents a summary chart showing the % wound remaining in the experimental groups in the diabetic mice model on day 7, 14 and 21 respectively in accordance with various embodiments.

FIG. 6 presents a summary chart showing the expression of Neutrophil elastase and MMP-8 in the granulation tissue collected around the wound area on day 7, 14 and 21 respectively in accordance with various embodiments.

It will be recognized that some or all of the figures are schematic representations for purpose of illustration in accordance with various embodiments.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

### Wound Dressing and Wound Healing Apparatus

As demonstrated in the experimental examples, biocompatible matrices prepared for controlled release of incorporated elafin protein achieve unexpected efficacy in treating wounds, in particular wounds in diabetic animals. In accordance with one embodiment of the present disclosure, therefore, provided is a wound dressing, comprising an effective amount of an elafin protein dispersed in a biocompatible matrix.

Elafin is also known as peptidase inhibitor 3 or skin-derived antileukoprotease (SKALP). In human, elafin is encoded by the PI3 gene. Elafin contains a WAP-type four-disulfide core (WFDC) domain, and is a member of the WFDC domain family. The human elafin sequence can be found in GenBank accession ID NP_002629 which is the preproprotein and includes 117 amino acid residues. Residues 61-117 constitute the mature elafin protein and is reproduced below as SEQ ID NO: 1.

| SEQ ID NO: | Sequence |
|---|---|
| 1 | AQEPVKGPVS TKPGSCPIIL IRCAMLNPPN |
| | RCLKDTDCPG IKKCCEGSCG MACFVPQ |

The elafin protein can be the mature protein of SEQ ID NO: 1 or one that further includes a signal peptide or other useful domains and sequences. In some embodiments, the elafin can also be a biological equivalent of SEQ ID NO: 1.

The term “a biological equivalent of a nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Biologically equivalent polynucleotides are those having the above-noted specified percent homology and encoding a polypeptide having the same or similar biological activity.

In some embodiments, one, two, three, four, five, or more amino acid residues can be substituted with conservative amino acid substitution. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

Conservative amino acid substitutions can also be any one shown in the following table.

| For Amino Acid | Substitution With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, L-Ser, |
| | D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

In some embodiments, the elafin protein has an amino acid sequence of SEQ ID NO: 1 or an amino acid sequence that has at least 75%, 80%, 85%, 90%, 95%, 95%, or 99% sequence identity to SEQ ID NO: 1. In some embodiments, the homologue retains the activity of the wild-type human elafin protein, such as the capability of inhibiting elastase, which can be readily measured with methods known in the art.

The amount of elafin protein in the matrix can be determined as needed. For instance, the amount of elafin protein can be determined based on how much elafin needs to be delivered to a wound per unit of area (e.g., per cm²). In some embodiments, the wound dressing includes from about 20 μg to about 500 μg elafin protein per cm² surface area of the wound dressing. In some embodiments, the wound dressing includes at least about 25, 30, 35, 40, 50, 60, 70, 80, 90, 100, 110, 120, 130, 140, 150, 160, 170, 180, 190, 200, 210, 220, 230, 240, or 250 μg elafin protein per cm² surface area of the wound dressing. In some embodiments, the wound dressing includes nor more than about 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110 or 100 μg elafin protein per cm² surface area of the wound dressing.

In some embodiments, the wound dressing includes from about 50 μg/cm² to about 200 μg/cm² elafin protein, from about 50 μg/cm² to about 200 μg/cm², from about 60 μg/cm² to about 180 μg/cm², from about 70 μg/cm² to about 160 μg/cm², from about 80 μg/cm² to about 140 μg/cm², from about 90 μg/cm² to about 120 μg/cm².

The biocompatible matrix can be prepared with various biocompatible materials such as polymers. Non-limiting synthetic polymers include, for example, polyphosphazenes, polyanhydrides, polyacetals, poly(ortho esters), polyphosphoesters, polycaprolactone, polyurethanes, polylactide, polycarbonates, and polyamides.

Polymers of biological sources can also be used, such as collagen. Collagen is the main structural protein in the extracellular space in the various connective tissues in animal bodies. As the main component of connective tissue, it is the most abundant protein in mammals, making up from 25% to 35% of the whole-body protein content. Depending upon the degree of mineralization, collagen tissues may be rigid (bone), compliant (tendon), or have a gradient from rigid to compliant (cartilage). Collagen, in the form of elongated fibrils, is mostly found in fibrous tissues such as tendons, ligaments and skin. It is also abundant in corneas, cartilage, bones, blood vessels, the gut, intervertebral discs, and the dentin in teeth. In muscle tissue, it serves as a major component of the endomysium. Collagen constitutes one to two percent of muscle tissue, and accounts for 6% of the weight of strong, tendinous muscles. The fibroblast is the most common cell that creates collagen.

At least 28 types of collagen have been identified and described. They can be divided into several groups according to the structure they form: fibrillar collagen (Type I, II, III, V, XI), and non-fibrillar collagen, which includes FACIT (Fibril Associated Collagens with Interrupted Triple Helices) (Type IX, XII, XIV, XVI, XIX), short chain (Type VIII, X), basement membrane (Type IV), multiplexin (Multiple Triple Helix domains with Interruptions) (Type XV, XVIII), MACIT (Membrane Associated Collagens with Interrupted Triple Helices) (Type XIII, XVII), and Other (Type VI, VII). The five most common types are Type I: skin, tendon, vascular ligature, organs, bone (main component of the organic part of bone); Type II: cartilage (main collagenous component of cartilage); Type III: reticulate (main component of reticular fibers), commonly found alongside type I; Type IV: forms basal lamina, the epithelium-secreted layer of the basement membrane; and Type V: cell surfaces, hair and placenta.

The biocompatible matrix can be made porous to allow controlled release of the elafin to a wound. In some embodiments, the average pore size is about 10 nm to about 100 μm, or from about 100 nm to about 10 μm. In some embodiments, the biocompatible matrix includes from about 5 mg/cm³ to about 100 mg/cm³ of its content (e.g., collagen). In some embodiments, the biocompatible matrix includes at least about 5, 10, 15, 20, 25, 30, 35, 40 mg of its content (e.g., collagen) per cm³ matrix. In some embodiments, the biocompatible matrix includes no more than about 100, 90, 80, 70, 60, 50, 40, 30, 25, 20 or 15 mg of its content (e.g., collagen) per cm³ matrix. In some embodiments, the biocompatible matrix comprises from about 15 mg/cm³ to about 30 mg/cm³ of its content (type 1 collagen).

The present disclosure also provides wound healing apparatuses that include the wound dressing. The wound healing apparatus may include a wound dressing of the disclosure disposed on a supporting material, such as an adhesive bandage.

### Preparation and Use

Methods of preparing and using the wound dressings and wound healing apparatuses of the disclosure are also provided. Biocompatible materials can be prepared with known in the art or obtained from commercial sources. For instance, collagen can be purified from animal tendon according to the established published protocols. A collagen solution can be prepared with a concentration of, e.g., 10 mg/ml, and is poured into a PDMS mold. The solution is allowed to dry in a sterile air flow chamber. The air drying results in soft collagen sponges which are sterilized before use.

The elafin protein or its biological equivalents can be expressed from a cell culture. For instance, E. coli, yeast, and mammalian cells can be used to express the protein. To incorporate the elafin protein to the biocompatible matrix, an elafin solution (e.g., 10 or 100 μg in 100 μl 10 mM phosphate buffer pH 7.4) can be absorbed on the collagen sponge and lyophilized to generate elafin-incorporated collagen composite matrices.

Methods of using the wound dressings or wound healing apparatuses of the disclosure are also provided. The methods can be useful for treating or improving the healing of a wound or ulcer. A wound is a sharp injury which damages the dermis of the skin.

An ulcer is a discontinuity or break in a bodily membrane that impedes the organ of which that membrane is a part from continuing its normal functions. Common forms of ulcers recognized in medicine include ulcer a discontinuity of the skin or a break in the skin (e.g., pressure ulcers, also known as bedsores; genital ulcer, an ulcer located on the genital area; ulcerative dermatitis, a skin disorder associated with bacterial growth often initiated by self-trauma; anal fissure, a.k.a. an ulcer or tear near the anus or within the rectum; and diabetic foot ulcer, a major complication of the diabetic foot), corneal ulcer, an inflammatory or infective condition of the cornea, mouth ulcer, an open sore inside the mouth (e.g., aphthous ulcer, a specific type of oral ulcer also known as a canker sore), peptic ulcer, a discontinuity of the gastrointestinal mucosa (stomach ulcer), venous ulcer, a wound thought to occur due to improper functioning of valves in the veins, stress ulcer, located anywhere within the stomach and proximal duodenum, ulcerative sarcoidosis, a cutaneous condition affecting people with sarcoidosis, ulcerative lichen planus, a rare variant of lichen planus, ulcerative colitis, a form of inflammatory bowel disease (IBD), and ulcerative disposition, a disorder or discomfort that causes severe abdominal distress, often associated with chronic gastritis. In one embodiment, the ulcer is a chronic ulcer. In one embodiment, the ulcer is a diabetic foot ulcer.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Elafin Incorporated Collagen for Treating Ulcers

Background:

This example tested to use elafin, a potent inhibitor of the elastase activity, incorporated into a collagen matrix for treating ulcers. The prepared collagenous sponge matrix incorporated with elafin resulted in slow release of elafin into the wounds upon contact. The collagen matrix served as the hemostat and prevented from further pathogenic invasion.

Methods:

A db/db diabetic mouse strain was used in this study. Standard full thickness wound of 0.8 cm was created on the dorsal back side of the mice. The mice were anesthetized using isoflurane and maintained under isoflurane anesthesia till the completion of the procedure. The hair was clipped using clippers and the skin was prepped using betadine solution. A sterile circular mold of 0.8 cm was placed over the skin and marked using a marker. A full thickness wound was created using a sterile scalpel and dressed according to the following study groups. The wound area was secured using silicon rings glued to the skin and further secured using skin sutures to prevent the wound contraction due to the shrinking process.

Preparation of Collagen Sponge:

The collagen solution was prepared in the following manner. 100 grams of bovine Achilles tendon collected from a slaughter house was thoroughly washed in plain water to free it from extraneous materials comprising of the surrounding tissues. The tendon tissue was washed well in water and chopped into smaller pieces, which were minced at 4 to 8° C. in a meat grinder. The minced material was then added to a scouring reagent comprised of 0.1% sodium laurel sulfate with vigorous stirring for 4 hours at 37° C. The scoured collagenous tissue was added to 0.1% solution of potassium peroxide after adjusting the pH to 10 and the stirring was continued for another 3 hours. The stock was then washed with water vigorously to remove loose non collagenous particles. The collagenous tissue was then treated with 2% pepsin solution at 4° C. with constant stirring, the pH was maintained at 2.5 by adding HCl. After 12 hrs, the pepsin treated collagenous mass was homogenized in a mechanical blender at 4-8° C. till a viscous solution was formed. The homogenate was diluted with 200 ml of milliQ water and 15 gm of potassium chloride was added with constant stirring. When a white precipitate of the collagen was formed, the reaction was stopped and centrifuged at 5000 rpm. The collagen was pelleted and the supernatant was discarded. The collagen precipitate was solubilized in 500 ml of acetic acid at pH3 while continuously stirring the solution for 90 minutes till a clear viscous solution of collagen was obtained. The homogenized collagen solution was dialyzed against 5 liters of 0.02M disodium hydrogen phosphate solution. The dialysate was centrifuged at 10000 rpm and the precipitate was redissolved in 500 mL of 0.5M acetic acid and dialyzed against 5 liters of milli Q water for 24 hrs at 4° C. to get pure collagen solution.

Preparation of Elafin:

The elafin was expressed using SHuffle T7 (New England Biolabs) E. coli cell strains. Cultures were grown on Terrific Broth (TB) growth medium with antibiotic (50 ug/mL kanamycin) at 30° C. to an OD600 of 0.5 at which time 0.5 mM IPTG was added to induce the production of the fusion proteins. The cultures were grown for a further 4 hours to a final OD600 of ˜1.3. Cells were pelleted at 6000 rpm for 10 minutes at 4° C. supernatant was discarded and pellet stored at −80° C.

The fusion protein was purified over HisPur Cobalt resin (Thermo Scientific) under native conditions using a gravity flow column. 4 ml of HisPur Cobalt resin was loaded into a glass column and allowed to settle forming a 2 ml resin bed. The column was equilibrated with two resin-bed volumes of equilibration/wash buffer. The lysate was mixed 1:1 with equilibration/wash buffer (50 mM sodium phosphate, 300 mM sodium chloride, 10 mM imidazole, pH7.4) and run over column collecting flow-through; the flow-through was reapplied to the column once. The column was washed with 2 resin volumes of equilibration/wash buffer; this step was repeated until the wash flow-through approached base line absorbance at 280 nm. The protein was eluted in five fractions containing 2 ml each, 10 ul of each fraction was run on a NuPAGE 10% Bis-Tris gel to determine protein elution, fractions 2 and 3 contained the majority of the eluted protein and these were combine for further use. Imidazole was removed by dialysis using a 3,000 MWCO slide-a-lyzer against 1 L of PBS at 4° C., twice, once for 4 hours and once overnight. Protein was quantified using the Pierce BCA Protein Assay Kit (Thermo Scientific).

The SUMO tag from SUMO elafin was cleaved off with SUMOstar protease by following the manufacturer's instructions. 0.5 mM DTT was added to the digestion reaction for optimal SUMOstar activity. The digest was eluted on HisPur Ni-NTA Spin column to remove both the SUMO fusion protein and SUMOstar protease. Protein concentration in the follow through was measured using the BCA protein assay kit.

Culture was grown as before. All buffers used for purification and other downstream applications were prepared in endotoxin free water. Cells were pelleted as before, but now resuspended in 40 ml Cobalt Binding Buffer (50 mM Na Phosphate, 300 mM NaCl, 10 mM Imidazole, pH 7.4), 8 M Urea, 0.1% Triton X-100 per liter of media, DNAse was added at 2 μg/mL and agitated for 30 minutes at 4 C. Insoluble protein was pelleted by centrifugation at 20,000 g's for 15 minutes. Cleared lysate was loaded onto an equilibrated HisTrap HP column (GE Life Sciences, 29-0510-21) charged with cobalt at 0.5 mL/min. The column was washed with 20 column volumes of Cobalt Binding Buffer, 8 M Urea, 0.1% Triton X-100 followed by 20 column volumes of Cobalt Binding Buffer, 8 M Urea running at 0.5 mL/min. Protein was eluted with 10 column volumes of Cobalt Elution Buffer (50 mM Na Phosphate, 300 mM NaCl, 150 mM Imidazole, pH 7.4), 8 M Urea. The eluted volume was dialyzed with PBS in a 3 k MWCO slide-a-lyzer (Thermo Scientific), 3 times at 500 times the eluted volume, to remove urea and imidazole. Protein was concentrated to ˜2 mg/ml using a 3 k MWCO Amicon Ultra.

Fabrication of the Elafin Incorporated Collagen Sponges.

Method 1: 10 gm of lyophilized bovine Achilles tendon (BAT) collagen was solubilized in 0.5M Acetic acid solution with constant stirring at 4° C. until a homogenous solution was obtained. The solubilized collagen solution was dialyzed against water for 24 hrs. The collagen solution was flooded with argon gas till a frothy collagen solution was obtained. This frothy mass was poured into PDMS mold to obtain dry collagen sponge in a sterile condition. The amount of collagen solution poured was maintained a constant to obtain sponge of uniform dimensions. Elafin at a concentration of 10 μg/ml in phosphate buffer (0.01M, pH 7.4) was slowly added evenly over the collagen sponge and allowed to penetrate the matrix. The dried matrix was lyophilized one more time to entrap the elafin solution.

Method 2: 10 gm of lyophilized BAT collagen was solubilized in 0.5M Acetic acid and dialyzed similar to method 1. Chondroitin sulfate at the ratio of 1:1 with mixed with the collagenous solution and stirred for 3 hours at 4° C. PEG was added to the mixture to give stability to the scaffold. Elafin 10 μg/ml in phosphate buffer (0.01M, pH 7.4) was mixed with the collagen, chondroitin sulfate matrix and allowed to stir at 4° C. until a homogenous solution was obtained. The homogenous solution was poured into PDMS mold and air dried at sterile conditions to obtain elafin incorporated collagen chondroitin sulfate matrix.

Method 3: A source of collagen (10 mL) thus obtained by method 1 was mixed with elafin 1 mL (10 μg/ml) in phosphate buffer (0.01M, pH 7.4) and constantly stirred for 24 hrs at 4° C. The solution was frothed with nitrogen gas with continuous stirring. The resulting solution was poured onto PDMS mold and lyophilized.

To evaluate the efficacy of the elafin, in vitro cell scratch using keratinocytes was performed to optimize the effective dose for the application in animal studies. Briefly, the keratinocytes were plated in a 6 well plate and allowed to reach 100% confluent. A 2004, sterile pipette tip was used to make a longitudinal scratch in the center of the plate. The cellular debris was removed by washing the plate once with the plated media. Elafin was added to the plating media at a concentration range of 1, 10 and 100 μg/ml and the plates were returned to the incubator. The control well had PBS instead of the elafin solution. Initial time point after scratch injury to the cells was captured at different lengths and the plates were observed every 6 hours for 48 hrs for the migration of the cells across the scratch. The complete closure of the scratch by the migrated cells were noted as the time required for closure of the wound and marked for the different conditions. The experiments were carried out in triplicates. The minimum dose required for the closure of the scratch wound in the cells were used for the fabrication of the elafin incorporated collagen scaffolds and tested in animals for wound healing.

Wound Healing Studies in dbdb Mice:

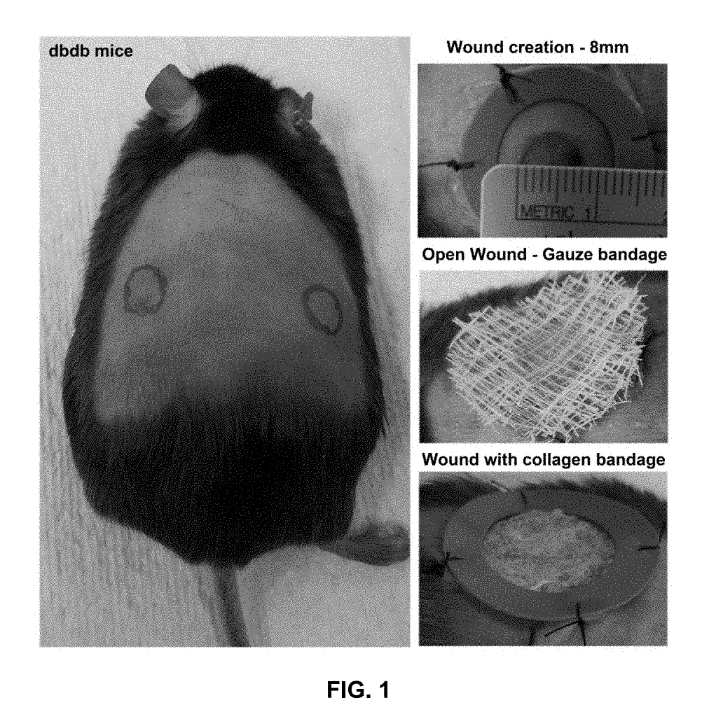

Adult female dbdb mice were used for the wound healing experiments with the elafin incorporated collagen scaffolds. Mice were anesthetized with 5% isoflurane (Isothesia, Henry Schein Animal Health, Dublin, Ohio) in 100% oxygen with a delivery rate of 51/minute until loss of righting reflex and mounted on a prone position in a surgical board. The anesthesia was maintained with 1 to 1.5% isoflurane throughout the surgical procedure. Body temperature was maintained using heating pads; respiration was monitored every 10 minutes. A pair of 0.8 cm circular punch wounds were created on the dorsal back as shown in FIG. 1. The wounded area was secured using silicon rings to prevent the shrinking of the wound area and to prevent natural healing in mice.

The wound area was treated with the following groups:
Group 1: Open wound covered with cotton gauze;
Group 2: Collagen Sponge;
Group 3: Open wound treated with 100 ug of Elafin;
Group 4: Open wound treated with bug of Elafin incorporated in to collagen sponge; and
Group 5: Open wound treated with 100 ug of Elafin incorporated in to the collagen sponge.

Wound Closure Analysis:

After surgery the wound area was monitored every day and photographed to survey the progress in wound healing. The wound closure was measured on day 7, 14, 21 and 30 to see the progress in healing. The wound closure was measured using the following equation:

$$\% Wound Closure = \frac{Area of Initial wound - Area of wound remaining}{Area of Initial wound} \times 100$$

Tissue Harvesting:

The granulation tissue around the wound area were collected on day 14, 21 and 30 days. A part of the tissue was collected for histopathological analysis and the other half of the tissue was cryoprotected for immunohistochemical analysis.

Western Blot Analysis:

Tissue samples were processed as previously described (Ahmed, E. et al., Exp Neurol 266 (2015) 42-54). Briefly, the skin was dissected, rapidly frozen on dry ice, and stored at −80° C. Skin tissue was collected from 100 μm slices cut on a cryostat (Leica CM1950, Leica Biosystems Inc, Buffalo Groove, Ill.). Skin samples were collected in 200 μl of lysis solution (Totally RNA, Ambion, Austin, Tex.). Care was taken to make sure that the tissue samples were collected from similar regions in all the samples analyzed. Protein concentration was estimated using Bradford reagent (Bio-Rad, Hercules, Calif.). 200 μg protein was precipitated with 100% methanol and centrifuged at 10000 g for 10 mins in a table top centrifuge. The pellet was re-suspended in 90% methanol and centrifuged for an additional 10 mins at 10000 g. The supernatant was discarded, and the pellet was air dried and dissolved in 400 μL of β-mercaptoethanol containing 2× Laemmli sample buffer (Biorad, Hercules, Calif.), for a final concentration of 0.5 mg/mL. These samples were stored at −80° C. until use. Samples were electrophoresed through 10% polyacrylamide gels and transferred on to Immobilon-P PVDF membranes (Millipore, Bedford, Mass.) and processed. Sample loading was counterbalanced across experimental groups. PVDF membranes were blocked with 5% skimmed milk powder for 1 hr at room temp and probed with the primary antibodies. Blots were washed with TBS-T to remove milk and incubated with primary antibodies neutrophil elastase and MMP-8 (Santacruz Biotechnology Inc) overnight at 4° C. and then with horse radish peroxidase-conjugated goat anti-rabbit or rabbit anti-mouse (H+L) secondary antibodies (Jackson ImmunoResearch Laboratories, West Groove, Pa.) at 1:5000 dilution. The blots were extensively washed with TBS-T after primary and secondary antibody incubations. Blots were developed using WesternBright ECL substrate (Advansta, Menlo Park, Calif.), following the manufacturer's instructions. This example used antibodies to quantify the levels of different proteins by Western.

Wound Closure:

A pair of 0.8 cm (8 mm) circular wound was created on either side of the back in the db/db mice as shown in FIG. 1. In the left panel, stencil marking was used to create a 0.8 cm wound on the dorsal side of the skin in db/db mice. The right upper panel shows the 0.8 cm full thickness wound created on the skin. The skin was secured with silicon rings glued using superglue and 4-0 nylon skin sutures. Scale shows the area of wound creation in cm. The right middle panel shows open wound dressed with standard cotton gauze bandage dressing and the right lower panel shows wound dressed using control collagen sponge matrix.

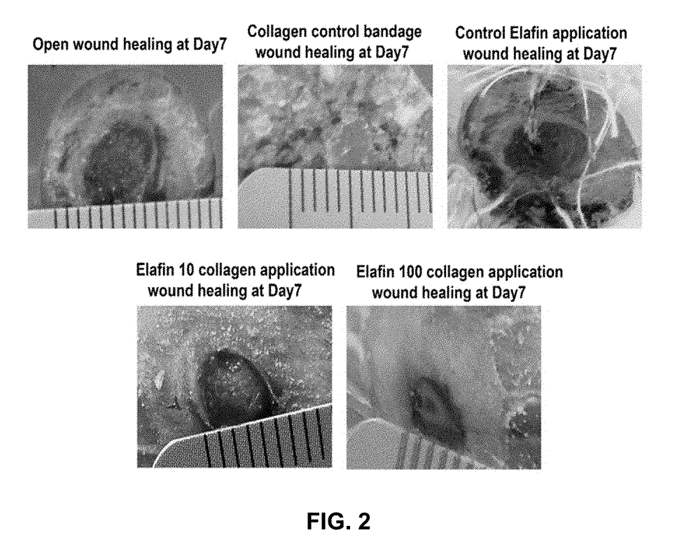

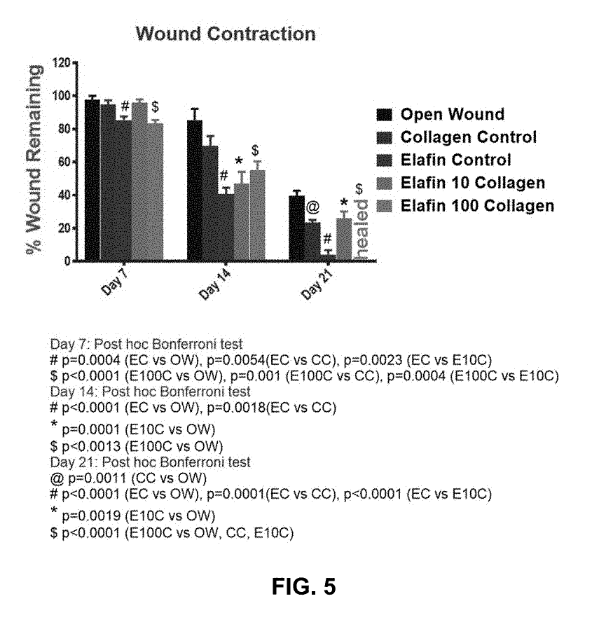

The wound closure was monitored every day for the total experimental period and photographed. The rate of wound closure was measured according to the formula described in the materials and methods section. The wound closure on days 7, 14 and 21 are shown in the FIG. 2-4. The rate of wound closure is shown in FIG. 5. On day 7, the Elafin alone and Elafin 100 μg incorporated sponge groups had significantly reduced wound area than the open wound, collagen sponge and Elafin 10 μg collagen sponge groups (the statistical significance is shown in FIG. 5).

Photomicrographs of wound healing studies in the mice at day 7 post wound creation are presented in FIG. 2. Photomicrographs on the upper left show the wound healing on day 7 in open wound group. The cotton gauze dressing was carefully removed to expose the underlying wound to digitally capture the remaining wound area. The scale insert shows the wound remaining after 7 days of wound creation. Wound remaining in Collagen control group after day 7 of wound creation (upper middle). The collagen sponge was partially removed to visualize the underlying wound without disturbing the wound area. Elafin control group after day 7 of wound creation (upper right). The cotton gauze was partially removed from the wound area to visualize the underlying wound area. The photomicrograph at lower left shows the wound healing at day 7 in Elafin 10 collagen sponge application group. The scale insert shows the wound area remaining after day 7. The photomicrograph at lower right shows wound healing at day 7 in Elafin 100 collagen sponge application group.

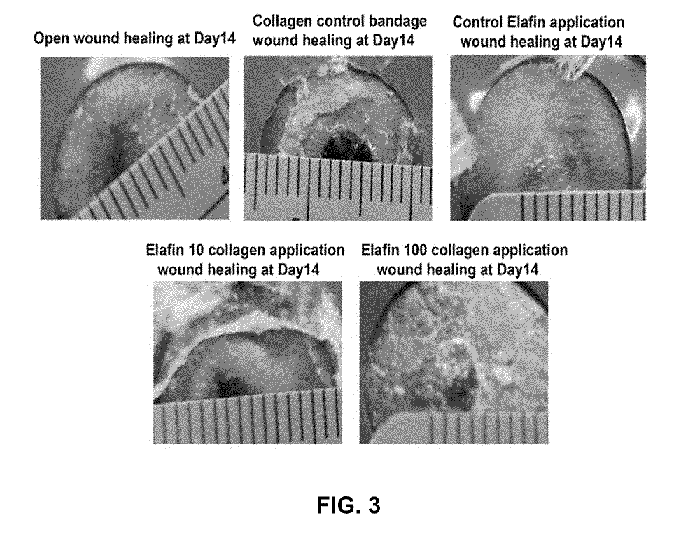

FIG. 3 presents photomicrographs of wound healing studies in db/db mice at day 14 post wound creation. At upper left, the photomicrograph shows the wound healing on day 14 in open wound group. The scale insert shows the wound remaining after 14 days of wound creation. The upper middle one shows wound remaining in Collagen control group after day 14 of wound creation. The collagen sponge was partially removed to expose the underlying wound without disturbing the wound area. The upper right figure shows Elafin control group after day 14 of wound creation. The cotton gauze was removed from the wound area to digitally capture the wound healing in this group. At lower left, the photomicrograph shows the wound healing at day 14 in Elafin 10 collagen sponge application group. The scale insert shows the wound area remaining after day 14. The lower right figure shows wound healing at day 14 in Elafin 100 collagen sponge application group. In all the collagen sponge application groups, the collagen matrix was secured to the periphery of the wound to prevent the removal of the collagen matrix. In order to access the wound area, the matrix needed to be removed.

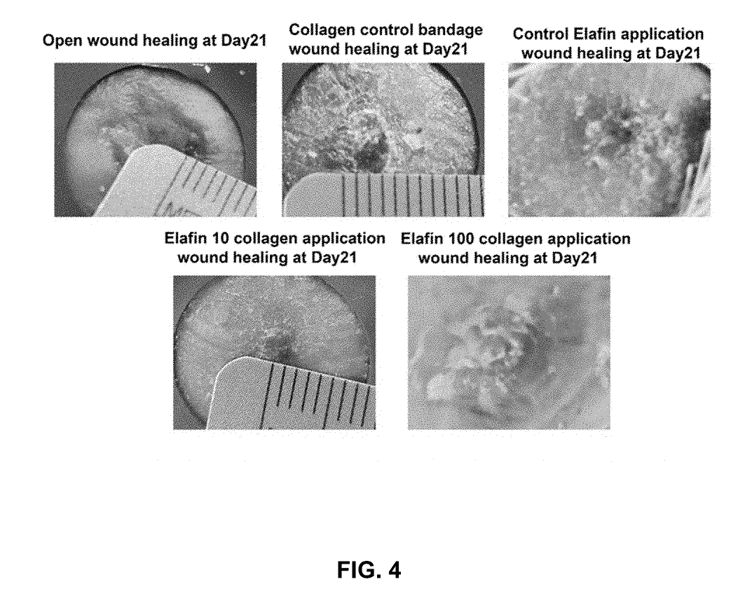

Photomicrographs of wound healing studies in db/db mice at day 21 post wound creation are presented in FIG. 4. At upper left, photomicrograph shows the wound healing on day 21 in open wound group. The upper middle figure shows wound remaining in Collagen control group after day 21 of wound creation. The upper right figure shows Elafin control group after day 21 of wound creation. At lower left, the photomicrograph shows the wound healing at day 21 in Elafin 10 collagen sponge application group. The lower right figure shows wound healing at day 21 in Elafin 100 collagen sponge application group. Complete closure of wound was seen at day 21 in Elafin 100 collagen sponge group compared to other treatment groups.

The granulation tissue around the wound area were collected on day 14, 21 and 30. A part of the tissue was collected to measure the expression of elastase and MMP-8 through western blot analysis on the aforementioned three days respectively.

The data from the above testing are summarized in FIG. 5. As shown, topical application of Elafin control and Elafin 100 Collagen sponge had significantly reduced wound area at day 7 of wound creation in db/db immunocompromised mice; at day 14, Elafin control, Elafin 10, Elafin 100 Collagen sponge containing bandage application at wound had significantly reduced wound area; and at day 21, complete healing and closure of the wound area was seen with Elafin 100 Collagen sponge bandages.

Levels of Elastase and MMP-8:

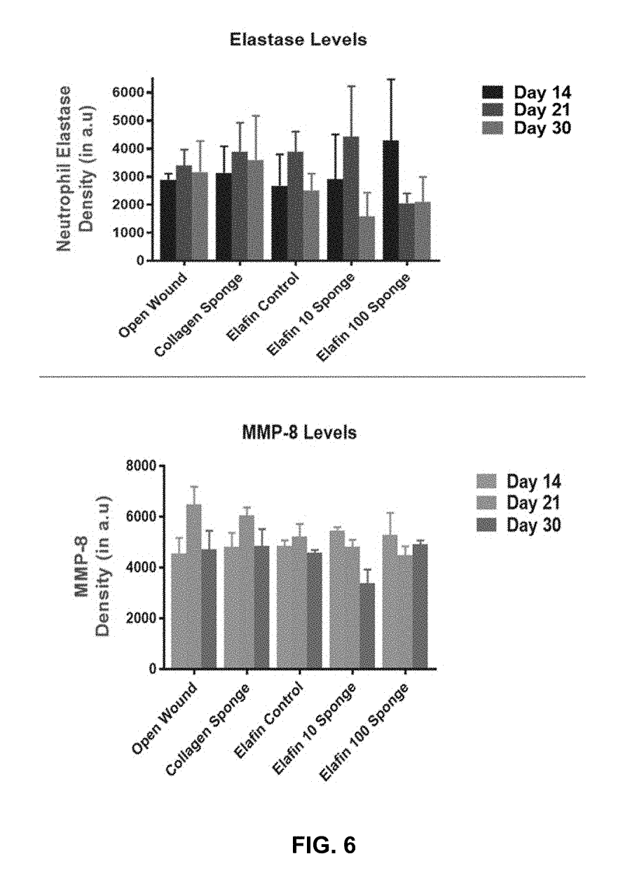

The levels of neutrophil elastase and MMP-8 were measured in the granulation tissue from the wound area on days 14, 21 and 30 and shown in FIG. 6. Elastase is the enzyme that breaks down the elastin in the skin and is thought to play a crucial role in the tissue remodeling where in it breaks down the elastin in the wound area resulting in the formation of scar. Elafin inhibits the action of elastin in the wound area and protects the wound environment from preventing the loss of elasticity and helps in faster regeneration of the skin tissue. MMPs on the other hand are matrix metalloproteinases which are secreted in the form of Pro-MMPs and upon cleavage becomes active MMP. MMP-8 is also known as neutrophil collagenase which is secreted by the neutrophils invading the wound area and results in tissue remodeling by breaking down the extracellular matrix protein collagen. The levels of these marker enzymes were studied on days 14, 21 and 30 when the tissue is regenerating for the complete closure of the wounds. The levels of elastase were moderate on day 14 of wound healing but increased significantly during day 21 in the groups except in the Elafin 100 ug incorporated collagen sponge groups showing that Elafin incorporated at higher levels in the collagen sponges were able to significantly inhibit the elastase activity in the wound area. The levels of elastase remained higher in the open wound and collagen sponge group than the Elafin alone or elafin 10 or 100 ug incorporated collagen sponges on day 30 showing that elafin is continuously released in the matrix which inhibits the activity of the elastase in the granulation tissue. On the other hand, MMP-8 levels also increased on day 21 in open wound and collagen sponge group following the pattern of elastase whereas in the elafin containing groups the increase was slightly lower. In the elafin bug incorporated collagen sponge group we saw a further reduction in the activity of MMP-8 than all the other experimental groups.

### CONCLUSION

The lower levels of elastase in the elafin 100 ug incorporated collagen groups positively correlates with the early wound closure in the elafin 100 ug incorporated collagen groups and shows that continuous slow release of elafin from the collagen matrix helps in faster healing of wound in a diabetic mice model of wound healing.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A wound dressing, comprising an effective amount of an elafin protein dispersed in a biocompatible matrix.

2. The wound dressing of claim 1, wherein the elafin protein comprises an amino acid sequence of SEQ ID NO: 1 or an amino acid sequence that has at least 90% sequence identity to SEQ ID NO: 1 and is capable of inhibiting elastase.

3. The wound dressing of claim 1, wherein the biocompatible matrix comprises a collagen.

4. The wound dressing of claim 3, wherein the collagen is type 1 collagen.

5. The wound dressing of claim 3, wherein the biocompatible matrix comprises from about 5 mg/cm³ to about 100 mg/cm³ collagen.

6. The wound dressing of claim 3, wherein the biocompatible matrix comprises from about 15 mg/cm³ to about 30 mg/cm³ type 1 collagen.

7. The wound dressing of claim 1, wherein the wound dressing comprises from about 20 μg/cm² to about 500 μg/cm² elafin protein.

8. The wound dressing of claim 7, wherein the wound dressing comprises from about 50 μg/cm² to about 200 μg/cm² elafin protein.

9. The wound dressing of claim 1, wherein the elafin protein is lyophilized.

10. The wound dressing of claim 1, wherein the elafin protein dispersed in a biocompatible matrix is disposed on a supporting material.

11. The wound dressing of claim 10, wherein the supporting material is an adhesive bandage.

12. A method of improving the healing of a wound, comprising applying a wound dressing on a wound, wherein the wound dressing comprises an effective amount of an elafin protein dispersed in a biocompatible matrix.

13. The method of claim 12, wherein the elafin protein comprises an amino acid sequence of SEQ ID NO: 1 or an amino acid sequence that has at least 90% sequence identity to SEQ ID NO: 1 and is capable of inhibiting elastase.

14. The method of claim 12, wherein the wound dressing comprises from about 20 μg/cm² to about 500 μg/cm² elafin protein.

15. The method of claim 12, wherein the elafin protein dispersed in a biocompatible matrix is disposed on a supporting material.

16. The method of claim 12, wherein the wound comprises an ulcer.

17. The method of claim 12, wherein the wound comprises chronic ulcer related to a diabetic condition.

18. A method of preparing a wound dressing, comprising:
obtaining a solution of an elafin protein;
obtaining a biocompatible matrix;
combining the solution of the elafin protein with the biocompatible matrix; and
drying the biocompatible matrix.

19. The method of claim 18, wherein the biocompatible matrix comprises a collagen and wherein the obtaining a biocompatible matrix step comprises isolating the collagen from a natural source.

20. The method of claim 19, wherein the combing step comprises the steps of:
solubilizing the biocompatible matrix;
mixing the biocompatible matrix with the solution of the elafin protein; and
forming the mixture in a mold under sterile conditions.


SEQUENCE LISTING (1 sequence)

SEQ ID NO: 1 - protein, 57 aa, Artificial Sequence
  Synthetic
AQEPVKGPVSTKPGSCPIILIRCAMLNPPNRCLKDTDCPGIKKCCEGSCGMACFVPQ